(19) United States
(12) Patent Application Publication
(10) Pub. No.: US 2010/0071687 A1
(43) Pub. Date: Mar. 25, 2010

(54) Nebulization Apparatus

(75) Inventors: Shu-Pin Hsieh, Taipei City (TW); Chien-Hua Lin, Taipei City (TW); Chang-Wei Lin, Kaohsiung City (TW); Andy Meng, Taipei City (TW); Yu-Chung Hsu, Taipei City (TW); Mei-Hui Huang, Tainan City (TW); Chia-Chen Huang, Kaohsiung County (TW); Chieh-Ming Hsiung, Taoyuan County (TW); Liang-Wei Liu, Taipei City (TW)

Correspondence Address: WPAT, PC;INTELLECTUAL PROPERTY ATTORNEYS, 2030 MAIN STREET, SUITE 1300, IRVINE, CA, 92614, US

(73) Assignee: MICRO BASE TECHNOLOGY CORPORATION, Taoyuan Hsien (TW)

(21) Appl. No.: 12/352,926

(22) Filed: Jan. 13, 2009

(30) Foreign Application Priority Data
Sep. 25, 2008 (TW) 097136992

(51) Int. Cl. A61M 11/00; B05B 17/04

(52) U.S. Cl. 128/200.14; 239/102.2

(57) ABSTRACT

A nebulization apparatus includes a nebulization plate, a piezoelectric driving device connected to the nebulization plate for vibrating the nebulization plate, a liquid droplet conveying pipe located below the nebulization plate, a water tank for containing the liquid droplet conveying pipe and an active water supply device connected to the liquid droplet conveying pipe and the water tank. The active water supply device supplies working liquid to the liquid droplet conveying pipe, such that the droplet is attracted by surface tension and attached onto the nebulization plate to form the camber water film to nebulize the working liquid.

### BACKGROUND OF THE INVENTION

1. Field of the Invention

The present invention relates to a nebulization apparatus, and more particularly to a nebulization apparatus having a liquid droplet conveying pipe for forming a camber water film by the surface tension between the liquid droplet conveying pipe and a nebulization plate to stabilize nebulizing a working liquid.

2. Description of the Related Art

In the methods of converting liquid into mist by the principle of vibration is used extensively for spraying a medicine or an air freshener, a piezoelectric ceramic vibrator having a vibration plate with striking holes on its surface is generally adopted for producing high-frequency slight vibration movements to form tiny particle mist molecules with a specific vector. With the Brownian movement, air is mixed with a scent to change the air quality or produce a medicine mist for the operation of an inhaling medical treatment. In addition to the applications for the scent and aerosol medicine, the aforementioned nebulization can be used for increasing the humidity indoor for countries or districts with a dry weather.

FIG. 1 illustrates a cross-sectional view of a conventional piezoelectric liquid nebulization apparatus. The piezoelectric liquid nebulization apparatus comprises a piezoelectric ceramic driver 11, a circular micro-hole plate 12, a water guide fiber 13 and a container 14. The driver 11 includes a through hole 111 therein, and a hemispherical surface 121 is disposed at the middle of the micro-hole plate 12, and a plurality of striking holes 122 are disposed on the hemispherical surface 121.

The working principle of this nebulization apparatus mainly extends the bottom of the water guide fiber 13 into the container 14 for performing a capillary action to suck a liquid out from the container 14. A water film 131 is formed at an upper end of the water guide fiber 13 due to the surface tension, and the water film 131 is attached onto the hemispherical surface 121. After the driver 11 is electrically connected to produce a vibration to drive a striking hole 122 of the micro-hole plate 12, the water film 131 is spluttered to convert the water film 131 into a gaseous state.

However, the water guide fiber 13 is a deformable and expandable absorber which produces different deformations according to the saturation level of absorbed liquid, and thus the deformation of the water film 131 and the hydrophilic force of the hemispherical surface are not uniform and cause a non-uniform excitation of nebulization.

After the water guide fiber 13 is expanded, a majority of vibration waves of the micro-hole plate 12 are absorbed by the water guide fiber 13 and lost. Most of the time, the micro-hole plate 12 is broken when it is compressed by the water guide fiber 13, and it results in the reduction of life of the nebulization apparatus.

Since the water guide fiber 13 sucks water by the capillary action, the water guide efficiency will be sensitive to the changes of the liquid level such that the nebulization rate will vary easily with time.

If the nebulization apparatus is doped with a medicine for a medical treatment purpose and the specific gravity of the medicine is not equal to that of the liquid, then the medicine will float or sink in the liquid, and the mixing effect will be insignificant or the medicine and solvent cannot be mixed uniformly. As a result, the excited mist will carry uniform medicine that may affect the medical treatment effect.

Since the absorption speeds of the water guide fiber 13 and the medical are not equal, a filtering effect is produced, such that the medicine is filtered by the water guide fiber 13 and remained in the liquid during the excitation of the mist, and the dosage carried by the mist will be insufficient.

In addition, TW Pat. No. I252130 discloses a draft structure of a high-frequency nebulization apparatus. FIG. 2 illustrates a cross-sectional view of another conventional nebulization apparatus. The excitation device 21 has a micro-hole plate 211, and the excitation device 21 is coupled to a floating body 221 of a floating unit 22 and parallel to the horizontal direction, and the floating body 221 floats at a liquid level 231 of a working liquid 23 in a container 24, such that the micro-hole plate 211 is close to the horizontal position of the liquid level 231. The working principle is to drive the excitation device 21 by electric power, so that the micro-hole plate 22 produces a high-frequency vibration acted onto the liquid level, and the water film of the liquid level receives the vibration energy and is decomposed to form a mist.

However, such nebulization apparatus has the following drawbacks. The micro-hole plate 211 is near the liquid level and forms a surface tension between the micro-hole plate 211 and the liquid level. Since the micro-hole plate 22 is attached by the surface tension, and most of the vibration energies of the micro-hole plate 22 transmitted from the excitation device are absorbed by the liquid level, and thus the nebulization efficiency becomes poor, or even worse, the nebulization effect will disappear.

Furthermore, the surface tension between the micro-hole plate 22 and the liquid level may cause the surface of the micro-hole plate 22 to accumulate a layer of water film that will affect the normal operation of the micro-hole plate and the effect of the high-frequency vibration.

### SUMMARY OF THE INVENTION

Therefore, one of objects of the present invention to provide a nebulization apparatus to overcome the shortcomings of the prior art that uses a water guide fiber for absorbing water, and the structural design of the water guide fiber causes an unequal hydrophilic forces of a water film and a nebulization plate and results in a non-uniform excitation of the nebulization.

To achieve the foregoing objective, the present invention provides a nebulization apparatus comprising a nebulization plate, a piezoelectric driving device, a liquid droplet conveying pipe, a water tank and an active water supply device. The piezoelectric driving device is connected to the nebulization plate for driving the nebulization plate to vibrate. The liquid droplet conveying pipe has a pipe opening and is located below the nebulization plate and contained in the water tank. The water tank contains a working liquid and the liquid droplet conveying pipe. The active water supply device is connected to the liquid droplet conveying pipe and the water tank. The active water supply device supplies a working liquid to the liquid droplet conveying pipe, such that the nebulization plate can nebulize the working liquid.

The working liquid is collected at the pipe opening to form a droplet, and the droplet is attracted by surface tension and attached onto the nebulization plate to form a camber water film.

Another objective of the present invention is to provide a nebulization apparatus comprising a nebulization plate, a piezoelectric driving device, a liquid droplet conveying pipe, a water tank, a passive water supply device and a casing. The piezoelectric driving device is coupled to the nebulization plate for driving the nebulization plate to vibrate. The liquid droplet conveying pipe has a pipe opening and is disposed below the nebulization plate. The water tank contains a working liquid, and is coupled to the liquid droplet conveying pipe. The passive water supply device controls the water tank to move and allow the working liquid to flow towards the liquid droplet conveying pipe, and a camber water film is formed between the pipe opening and the nebulization plate, such that the nebulization plate can nebulize the working liquid. The casing contains the passive water supply device and the water tank.

The passive water supply device includes a spring structure pressed against the water tank and connected to the casing for controlling the water tank to move in a vertical direction to maintain the height difference between the liquid level of the working liquid and the pipe opening of the liquid droplet conveying pipe.

The water tank further includes a plurality of micro ventilation holes disposed at the top of the water tank for allowing air to enter into the water tank to stabilize the air pressure of the water tank.

In summation of the description above, the nebulization apparatus of the invention has one or more of the following advantages:

(1) The nebulization apparatus supplies the working liquid at the pipe opening of the liquid droplet conveying pipe, and the working liquid is collected to form a droplet, and the droplet is attracted by surface tension and attached onto the nebulization plate to form a camber water film, so as to prevent a non-uniform nebulization that is generally occurred in a prior art nebulization apparatus. Since the prior art uses a water guide fiber or a capillary to guide water, and after the water guide fiber or capillary is deformed, the water film is expanded, and too much working liquid will remain on the nebulization plate.

(2) The nebulization apparatus provides the active water supply device or the passive water supply device to maintain the height difference between the liquid level and the pipe opening to stabilize the nebulization rate.

(3) The liquid droplet conveying pipe of the nebulization apparatus does not produce a filtering effect, so that when the nebulization apparatus is used for the medical treatment purpose, the nebulized medicine can maintain its uniformity to give the normal medical effect.

(4) The liquid droplet conveying pipe of the nebulization apparatus will not be deformed or expanded to press against the nebulization plate, so as to prevent the nebulization plate from aging or cracking that will reduce the life of the nebulization apparatus.

### BRIEF DESCRIPTION OF THE DRAWINGS

The subject matter regarded as the invention is particularly pointed out and distinctly claimed in the concluding portion of the specification. The invention together with features and advantages thereof may best be understood by reference to the following detailed description with the accompanying drawings in which:

FIG. 1 is a cross-sectional view of a conventional piezoelectric liquid nebulization apparatus;

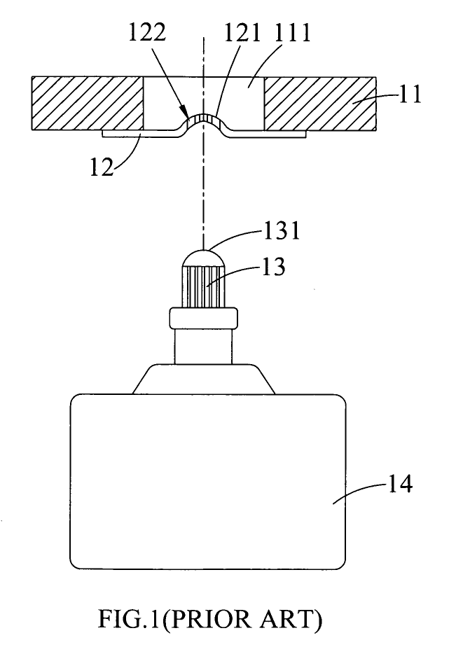

FIG. 2 is a cross-sectional view of another conventional piezoelectric liquid nebulization apparatus;

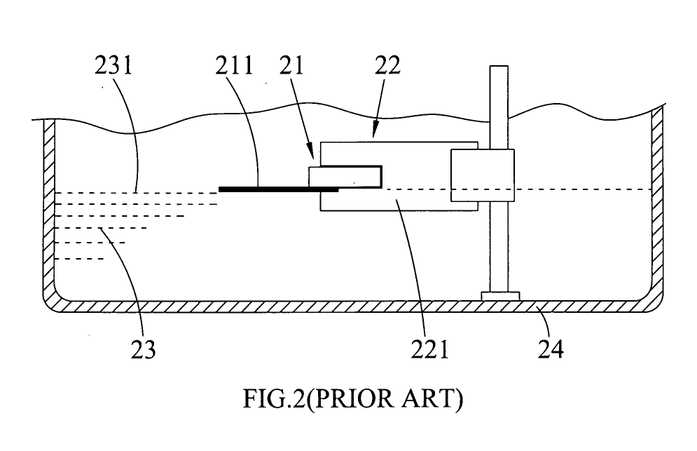

FIG. 3 is a cross-sectional view of a nebulization apparatus in accordance with a first preferred embodiment of the present invention;

FIG. 4 is a flow chart of movements of a nebulization apparatus in accordance with a first preferred embodiment of the present invention, showing that a droplet is formed at a pipe opening by surface tension;

FIG. 5 is a schematic view of geometric cross-sections of a pipe opening of a nebulization apparatus in accordance with a first preferred embodiment of the present invention;

FIG. 6 is a schematic view of a nebulization apparatus in accordance with a first preferred embodiment of the present invention, showing that a water refiller is located at the nebulization apparatus for supplying a working liquid; and

FIG. 7 is a cross-sectional view of a nebulization apparatus in accordance with a second preferred embodiment of the present invention.

### DETAILED DESCRIPTION OF THE PREFERRED EMBODIMENTS

The present invention relates to a nebulization apparatus. While the specifications describe at least one embodiment of the invention considered best modes of practicing the invention, it should be understood that the invention can be implemented in many ways and is not limited to the particular examples described below or to the particular manner in which any features of such examples are implemented.

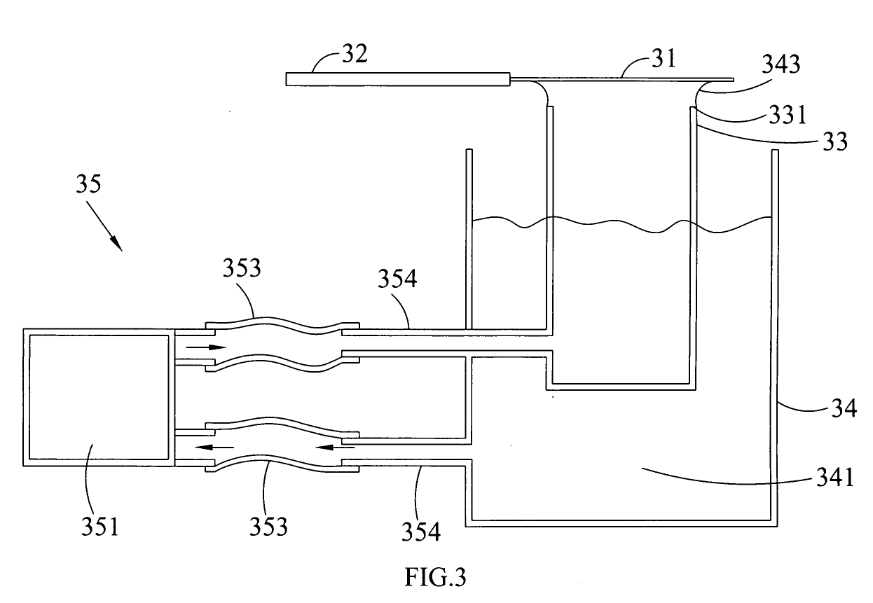

FIG. 3 illustrates a cross-sectional view of a nebulization apparatus in accordance with a first preferred embodiment of the present invention. The nebulization apparatus comprises a nebulization plate 31, a piezoelectric driving device 32, a liquid droplet conveying pipe 33, a water tank 34 and an active water supply device 35. The piezoelectric driving device 32 is made of a glass or ceramic material and provided for driving the nebulization plate 31 to produce vibrations after the piezoelectric driving device 32 is electrically connected. The liquid droplet conveying pipe 33 has a pipe opening 331 disposed below the nebulization plate 31 and contained in the water tank 34. The water tank 34 contains a working liquid 341, and the working liquid has a liquid level 343. The active water supply device 35 is coupled to the liquid droplet conveying pipe 33 and the water tank 34.

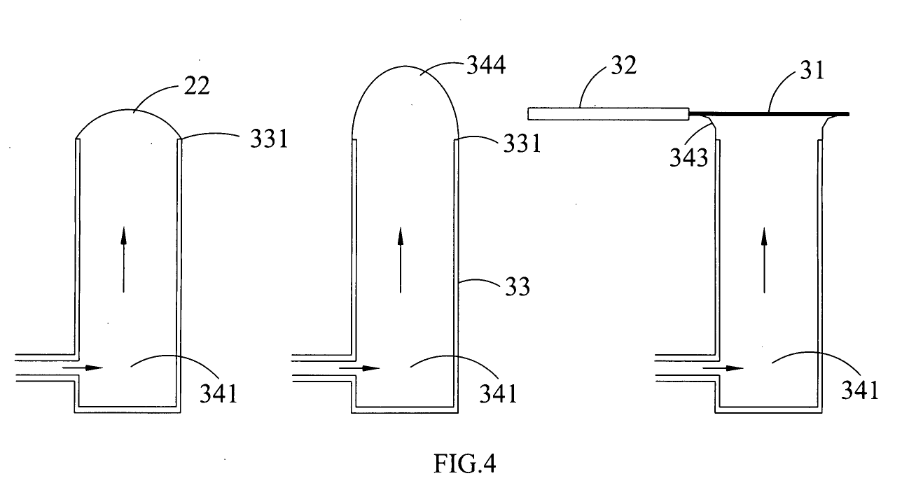

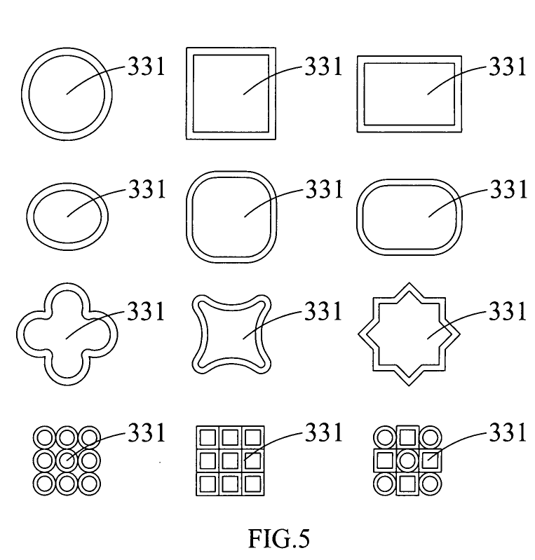

FIG. 4 illustrates a flow chart of movements of a liquid droplet conveying pipe in accordance with a first preferred embodiment of the present invention. The hemispherical droplet 344 is formed naturally at the pipe opening 331 by the surface tension when the working liquid 341 overflows from the pipe opening 331. Since the distance between the nebulization plate 31 and the pipe opening 331 is approximately equal to or greater than half of the height (50%˜80%) of the hemispherical droplet 344, an end of the droplet 344 will be attached onto the nebulization plate 31 by surface tension to form a camber water film 343 naturally after the overflowed droplet 344 reaches a specific size. In addition, the cross-section of the pipe opening 331 of the liquid droplet conveying pipe 33 matches the operating area of the nebulization plate 31 and can be of a rectangular, circular, star-shaped or elliptic shape, or a geometric shape of any combination of the above as shown in FIG. 5.

The active water supply device 35 includes a pump 351 (or a motor), two connecting pipes 353 connected to the pump 351, and two conveying pipe devices 354 connected to the liquid droplet conveying pipe 33 and the water tank 34 respectively. An end of the conveying pipe device 354 is connected to the connecting pipe 353, such that the pump 351 can supply water to the liquid droplet conveying pipe 33 steadily through the connecting pipe 353 and the conveying pipe device 354. If the water supplied by the pump 351 is too much and exceeds the attaching force of the surface tension, a portion of water will overflow to the pipe opening 331 and flow back into the water tank 34, so that the camber water film 343 will remain unchanged (without deformations), and no excessive working liquid 341 remains at the surface of the nebulization plate 31, and the vibration energy of the nebulization plate 31 is absorbed by the remained working liquid 341 that may affect the nebulization performance. The working liquid 341 flowing back into the water tank can be guided by the conveying pipe device 354 and the connecting pipe 353 to the pump 351 for recycles and reuses.

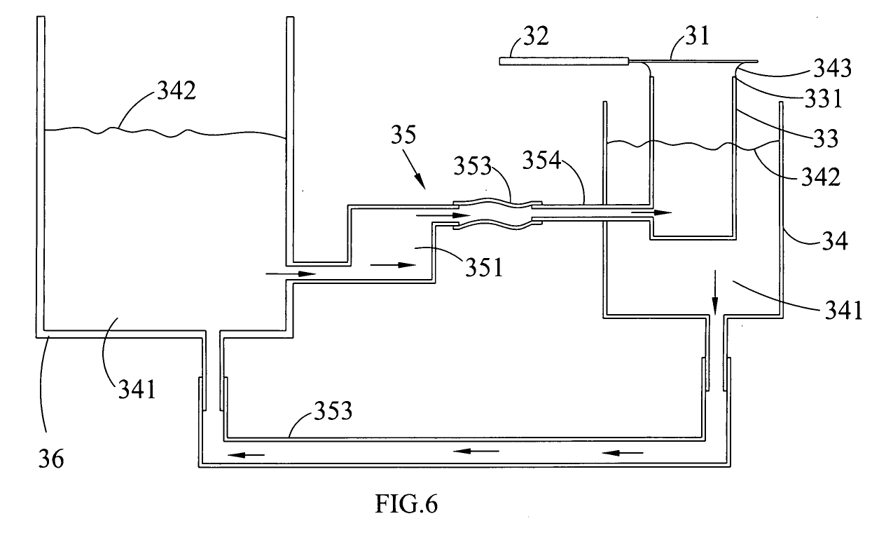

In addition, a water refiller 36 can be located at the nebulization apparatus. With the connecting pipe 353 connected to the water tank 34 and the pump 351, the working liquid 341 can be supplied sufficiently and stably or can satisfy different design requirements as shown in FIG. 6, wherein the working liquid 341 is a liquid medicine, a scented essential oil or water for humidifying a room.

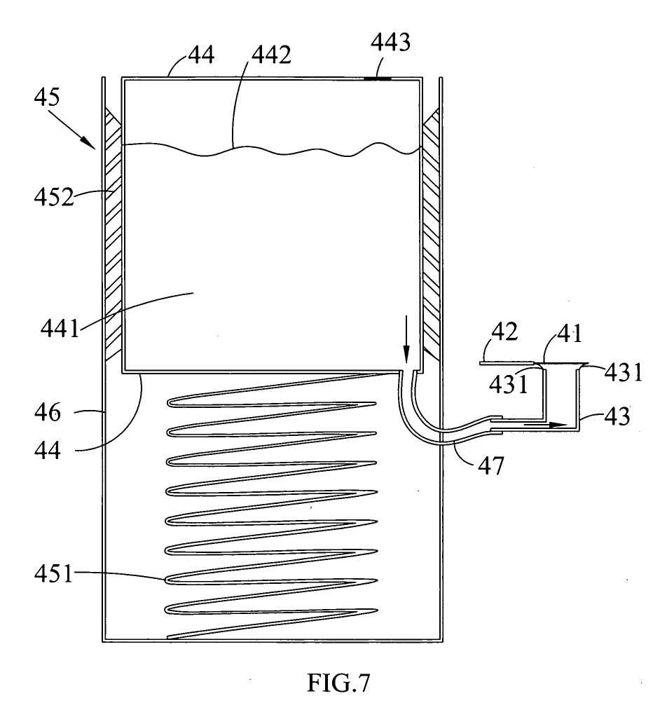

FIG. 7 illustrates a cross-sectional view of a nebulization apparatus in accordance with a second preferred embodiment of the present invention. The nebulization apparatus comprises a nebulization plate 41, a piezoelectric driving device 42, a liquid droplet conveying pipe 43, a water tank 44, a passive water supply device 45 and a casing 46. The working liquid 441 is contained in the water tank 44 and forms a liquid level 442, and the liquid droplet conveying pipe 43 comes with a pipe opening 431. The water tank 44 and the liquid droplet conveying pipe 43 are connected by a connecting pipe 47. The structure and function of the nebulization plate 41, the piezoelectric driving device 42, the liquid droplet conveying pipe 43 and the water tank 44 are the same as those of the first preferred embodiment, and thus will not be described here again.

The difference of this preferred embodiment from the first preferred embodiment resides on that the passive water supply device 45 includes a spring structure 451 and a plurality of slide rails 452. The water tank 44, the spring structure 451 and the slide rail 452 are installed in the casing 46. An end of the spring structure 451 is pressed against the bottom of the water tank 44, and another end of the spring structure 451 is pressed against and coupled to the casing 46. As the weight of the water tank 44 varies, the spring structure 451 is used to control the water tank 44 to move in the vertical direction to maintain the height difference between the liquid level 442 and the pipe opening 431 of the liquid droplet conveying pipe 43, in order to maintain a constant liquid level pressure difference, and allow the working liquid 441 to overflow from the pipe opening 431 stably. After the working liquid 441 is overflowed from the pipe opening 431, the surface tension between the working liquid 441 and the pipe opening 431 is acted at the pipe opening 431 to collect the working liquid 441 to form a droplet 432. Since the nebulization plate 41 is proximate to the pipe opening 431, the droplet 432 will be attached onto the nebulization plate 41 to form a camber water film 433, after the droplet 432 grows to a specific size.

The slide rails 452 are installed separately on both sides of the water tank 44. If the spring structure 451 drives the water tank 44 to move in a vertical direction, the slide rail 452 slides in the casing 46 to maintain a smooth movement of the water tank.

For instance, if the nebulization apparatus keeps nebulizing the working liquid 441, the working liquid 441 in the water tank 44 will be decreased, and the weight of the water tank 44 will be reduced, so that the resilience of the spring structure 451 will press against the water tank 44 to move the water tank 44 upward, and the gravity and elastic balance can be maintained. The height difference between the liquid level 442 and the pipe opening 431 can be maintained without increasing or decreasing the working liquid 441. Different elasticity coefficients can be used for the spring structure 451 to fit the water tanks 44 of different sizes.

The water tank 44 further includes a plurality of micro ventilation holes 443 disposed at the top of the water tank 44, and the hole diameter is approximately equal to 1 um to 200 um. The micro ventilation hole 443 is provided for limiting the air flow of the outside air entering into the water tank 44 to stabilize the air pressure in the water tank 44 to control the overflow of the working liquid at the pipe opening, so that the nebulization apparatus can achieve the effect of spraying a tiny amount of liquid.

What is claimed is:

1. A nebulization apparatus, comprising:
a nebulization plate;
a piezoelectric driving device, coupled to the nebulization plate, capable of driving the nebulization plate to vibrate;
a liquid droplet conveying pipe, having a pipe opening and located below the nebulization plate;
a water tank, for containing the liquid droplet conveying pipe and a working liquid; and
an active water supply device, connected to the liquid droplet conveying pipe and the water tank;
wherein the active water supply device supplies the working liquid to the liquid droplet conveying pipe, and a camber water film is formed between the pipe opening and the nebulization plate, such that the nebulization plate can nebulize the working liquid.

2. The nebulization apparatus of claim 1, wherein the working liquid is collected at the pipe opening to form a droplet, and the droplet is attracted by a surface tension to attach onto the nebulization plate to form the camber water film.

3. The nebulization apparatus of claim 1, wherein the active water supply device is a motor or a pump.

4. The nebulization apparatus of claim 3, further comprising a water refilling tank connected to the active water supply device and the water tank for refilling the loss of the working liquid nebulized by the nebulization plate.

5. The nebulization apparatus of claim 1, wherein the pipe opening has a substantially rectangular, circular, star-shaped or elliptic cross-section.

6. The nebulization apparatus of claim 1, wherein the working liquid is a liquid medicine, a scented essential oil or water.

7. A nebulization apparatus, comprising:
a nebulization plate;
a piezoelectric driving device, coupled to the nebulization plate, capable of driving the nebulization plate to vibrate;
a liquid droplet conveying pipe, having a pipe opening and located below the nebulization plate;
a water tank, for containing a working liquid, and coupled to the liquid droplet conveying pipe;
a passive water supply device, capable of controlling movement of the water tank to allow the working liquid to flow towards the liquid droplet conveying pipe, wherein a camber water film is formed between the pipe opening and the nebulization plate, such that the nebulization plate can nebulize the working liquid; and
a casing, for containing the passive water supply device and the water tank.

8. The nebulization apparatus of claim 7, wherein the working liquid is connected at the pipe opening to form a droplet, and the droplet is attracted by a surface tension to attach onto the nebulization plate to form the camber water film.

9. The nebulization apparatus of claim 7, wherein the passive water supply device includes a spring structure pressed against the bottom of the water tank and coupled to the casing, for controlling the water tank to move in a vertical direction to maintain the height difference between a liquid level of the working liquid and the pipe opening of the liquid droplet conveying pipe.

10. The nebulization apparatus of claim 9, wherein the passive water supply device further comprises a plurality of slide rails installed on both sides of the water tank, such that when the spring structure is operated, the plurality of slide rails slide on the casing to maintain a smooth movement of the water tank.

11. The nebulization apparatus of claim 7, wherein the water tank further includes a plurality of ventilation holes disposed at the top of the water tank, for allowing air to enter the water tank for stabilizing the air pressure of the water tank.

12. The nebulization apparatus of claim 7, wherein the pipe opening has a rectangular, circular, star-shaped or elliptic cross-section.

13. The nebulization apparatus of claim 7, wherein the working liquid is a liquid medicine, a scented essential oil or water.
